(19) United States
(12) Patent Application Publication
(10) Pub. No.: US 2025/0161533 A1
(43) Pub. Date: May 22, 2025

(54) Allograft-Polymer Hybrids, Methods of Making and Methods of Use

(71) Applicant: University of Maryland, Baltimore, Baltimore, MD (US)

(72) Inventors: Tao Lowe, Hummelstown, PA (US); Michael Hartman, Lewisberry, PA (US)

(73) Assignee: University of Maryland, Baltimore, Baltimore, MD (US)

(21) Appl. No.: 18/840,245

(22) PCT Filed: Feb. 24, 2023
(86) PCT No.: PCT/US2023/063202

§ 371 Date: Aug. 21, 2024

Related U.S. Application Data
(60) Provisional application No. 63/313,551, filed on Feb. 24, 2022.

(51) Int. Cl. A61L 27/36; A61L 27/38; A61L 27/44; A61L 27/52; A61L 27/54; A61L 27/56; A61L 27/58

(52) U.S. Cl. CPC A61L 27/3695; A61L 27/3834; A61L 27/44; A61L 27/52; A61L 27/54; A61L 27/56; A61L 27/58; A61L 2300/414; A61L 2430/34

(57) ABSTRACT

Provided herein are bioresorbable, bioregenerative composite materials and compositions to regenerate tissues or organs to repair tissue or organ defects and constructs printed from the same as 3D structures shaped and sized to fill a tissue or organ defect. The composite materials are bioresorbable polymers, for example, polycaprolactone, and a bioresorbable graft material, for example, demineralized bone matrix. Also provided are methods for making the composite materials and compositions and methods for regenerating tissues or organs and repairing a tissue or organ defect using the constructs.

### CROSS-REFERENCE TO RELATED APPLICATIONS

This international application claims the benefit of priority under 35 U.S.C. § 119 (e) of provisional application U.S. Ser. No. 63/313,551, filed Feb. 24, 2022, the entirety of which is hereby incorporated by reference.

### BACKGROUND OF THE INVENTION

### Field of the Invention

The present invention relates to the fields of molecular biology, biomedical engineering, biomaterial, nanotechnology, drug delivery, 3D-printing, tissue engineering, regenerative medicine, and bone physiology. More particularly, the present invention relates to allograft-polymer hybrid constructs for patient-specific and defect-site-specific, human and animal tissue and organ particularly bone regeneration.

### Description of the Related Art

Over a billion people in the world currently suffer from bone fractures and/or bone diseases, and the prevalence will increase as the global population ages (1,2). Critical-sized bone defects, which can be attributed to trauma, tumor resection, infection, developmental deformities and degenerative diseases, have minimal regenerative capabilities and can cause life-long debilitating pain and disability, which significantly negatively impact the quality of life of the patients (3,4).

The current treatments for the critical-sized bone defects include gold-standard autologous bone transplantation, allogeneic bone grafting, synthetic bone graft substitutes, distraction osteogenesis, and induced membrane technique (3,4). The global bone grafts and substitutes market was valued at $2.9 billion in 2021, and is projected to reach $5 billion by 2030 (5). These treatments, however, have issues of donor site morbidity, shortage of donor tissues, high cost, increased operative time, nonunion, malunion, fracture, infection, immunogenic reactions, and/or transfer of diseases.

Advances in tissue engineering, biomaterial design, and bioprinting create opportunities to fabricate patient specific and functional bone substitutes. Tissue engineering using stem cells, biomaterials and biologically active molecules can provides 3D structural and mechanical support, and molecular signals that induce differentiation of stem cells into osteoblasts (6,7). The biomaterials used as bone substitutes include inorganic materials: ceramics, bioactive glass, tricalcium phosphate, and hydroxyapatite, which have strong mechanical strength but are brittle; naturally derived organic polymers: alginate, gelatin, collagen, fibrin, chitosan, hyaluronic acid and silk, which have good biocompatibility and biorecognition but weak mechanical strength; synthetic biodegradable organic polymers: polylactic acid (PLA), polyglycolic acid (PGA), poly lactic acid-co-glycolic acid (PLGA) and poly(ε-caprolactone) (PCL), which have moderate mechanical strength but good viscoelasticity and controlled biodegradability with PCL having the highest mechanical strength and the slowest hydrolytically degradation rate; and hybrids of the inorganic and organic biomaterials, which have combined mechanical, viscoelastic, biodegradable and biorecognizable strengths of the two types of materials (3,4,7-10). However, none of these materials can provide crucial properties that can match with the tailored shapes, and mechanical and biological context of real bone (3,4,7-11). These biomaterials are osteoconductive to allow bone ingrowth but not osteoinductive and osteogenic (3,4,7-10).

Bone morphogenic proteins 2 and 7 (BMP-2 and BMP-7), platelet derived growth factor (PDGF), transforming growth factor-b1, insulin-like growth factor 1, vascular endothelial growth factor and fibroblast growth factor are major osteoinductive growth factors that regulate bone remodeling cascade and play crucial role in bone formation and repair (3,6,12,13). However, these growth factors have issues of low stability and short half-life (6) and long-term release of these intact growth factors are needed. Demineralized bone matrix (DBM) composed of 93% collagen and 5% growth factors is an osteoconductive and weak osteoinductive allograft. It is available in large quantity, avoids donor site morbidity, and has less immunogenic than autograft. DBM allografts, however, cannot be used as a stand-alone bone substitute as autografts and synthetic bone graft substitutes due to their rapid resorption rate and weak mechanical strength (10,11). In addition, demineralized bone matrix allografts are not osteogenic. Mesenchymal, dental pulp, induced pluripotent, embryonic, adipose tissue-derived and muscle-derived stem cells have distinct capability to differentiate into osteogenic lineages, and thus have become important and widely used for bone tissue engineering (3,6,14).

Traditionally, cells for bone tissue engineering were simply allowed to aggregate into 3D pellets or supported in a 3D porous biomaterial scaffold developed by solvent casting, particle-leaching, nonwoven or electrospun nanofibrous meshes. These cell encapsulation methods cannot control the scaffold pore and geometry to replicate the complex tissue architecture. With the invention of 3D bioprinting technology since 2003 (15), 3D-biomaterial constructs with controlled porosity, geometry and architecture can be fabricated with uniformity, which opens a new avenue for fabrication of patient-specific and defect-site-specific constructs for bone tissue engineering (16-20).

The prior art is deficient in bone substitutes that mimic the natural bone healing process to regenerate functional bones. The present invention fulfills this need and desire in the art.

### SUMMARY OF THE INVENTION

The present invention is directed to a composite material for regeneration of a tissue or an organ, such as, but not limited to, bone. The composite material is a mixture of a bioresorbable polymer and a bioresorbable graft material.

The present invention is also directed to a bioresorbable construct to repair a defect in a tissue or an organ. The bioresorbable construct is a 3D structure printed from the composite material described herein. The present invention is directed to a related bioresorbable construct where the 3D structure further comprises a growth factor, a gene or an additive loaded therein with or without a biodegradable nanocarrier or cells, or a combination thereof. The present invention is directed to another related bioresorbable construct where the 3D structure further comprises a sensor, an imaging probe, an imaging dye, or a semiconductor, or a combination thereof.

The present invention is directed further to a method for repairing a bone defect in a subject in need thereof. In the method the bioresorbable construct described herein is printed as the 3D structure comprising a plurality of interconnected pores thereon and dimensions corresponding to the defect in the tissue or the organ defect. The 3D structure is loaded with stem cells, said 3D structure optionally comprising a growth factor, a gene, an additive, tissue-specific cells, a sensor, an imaging probe, an imaging dye or a semiconductor, or a combination thereof; said growth factor, said gene or said addition optionally loaded into a biodegradable nanocarrier. The bioresorbable construct is implanted into the defect of the tissue or the organ where the stem cells contained therein are induced to differentiate into osteoblasts, thereby regenerating the bone in the subject.

The present invention is directed further still to a method for preparing a composite of a bioresorbable polymer and a bioresorbable osteoconductive graft material suitable for 3D printing. In the method a solid powder mixture of a resorbable polymer and an osteoconductive graft material is prepared and milling the solid powder mixture is milled to reduce a particle size of the osteoconductive graft material, thereby producing the composite. The present invention is directed to a related method further comprising heating the composite to mold into pellets and extruding the pellets into filaments suitable for 3D printing.

The present invention is directed further still to a composite of a bioresorbable polymer and a bioresorbable graft material prepared by the method described herein.

The present invention is directed further still to a hybrid bioregenerative composition. The hybrid bioregenerative composition is a composite of polycaprolactone and an allograft, autograft, or xenograft.

The present invention is directed further still to a tissue or organ regenerative construct. The tissue or organ regenerative construct is a composite of polycaprolactone and human demineralized tissue or organ matrix printed as a 3D structure with a plurality of interconnected pores and dimensions corresponding to a defect in the tissue or organ. Each of the interconnected pores in the plurality comprises stem cells and, optionally, a growth factor, a gene, or an additive loaded with or without a biodegradable nanocarrier, tissue-specific cells, a sensor, an imaging probe, an imaging dye, or a semiconductor, or a combination thereof.

The present invention is directed further still to a method for regenerating a tissue or an organ in a subject in need thereof. In the method the tissue or organ regenerative construct described herein is implanted into the defect in the tissue or organ, where the stem cells contained therein are induced to differentiate into tissue-specific cells, thereby regenerating the tissue or the organ in the subject.

Other and further aspects, features, benefits, and advantages of the present invention will be apparent from the following description of the presently preferred embodiments of the invention given for the purpose of disclosure.

### BRIEF DESCRIPTION OF THE FIGURES

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1 shows that 3D-printed constructs made of allograft and PCL and designed for insulin release have high compression Young's modulus. Allevi 3 bioprinter (3D Systems) was utilized to print constructs made of 10% allograft/90% PCL with pore sizes 0.1 or 0.5 mm. A Dynamic Mechanical Analyzer (DMA, Instron 5565) was used to measure the compression Young's moduli of the constructs. Each bar represents the mean±s.d. of 4 replicates. *, p<0.05.

FIG. 2 shows that nanogels prolong model protein insulin release. 20 mg insulin-loaded nanogels were dispersed in 1 mL PBS (pH 7.4) in a release device (Spectra/Por® Float-A-Lyzer G2, 50K MWCO). The release device was then immersed in 20 mL of PBS (pH 7.4) release medium in a 50 ml conical centrifuge tube at 37° C. At selected time points, the release media were collected and quantified to measure the amounts of insulin by mass spectroscopy. Data were presented by accumulated insulin release amount at t time. Each bar represents the mean±s.d. of 4 replicates.

FIGS. 3A-3F show CADed constructs, of dimension 6 mm in diameter by 1 mm in height, are uploaded as STLs having the resultant appearances of interconnected pores with 500 μm pore size along the top and walls (FIG. 3A), and basic cylindrical construct for which infill distance can be tuned by slic3r settings to have desired pore sizes of 500 μm (FIG. 3B). Representative allograft-PCL construct with interconnected pores along the top and walls (FIG. 3C) as well as porous cylindrical construct of PCL-allograft printed with as high as 45 wt % allograft (FIG. 3D). Microscopic images of printed constructs for the interconnected pore construct and basic cylindrical construct show consistent and open pore structures with minimal stringing (FIGS. 3E-3F).

FIG. 4 shows the effect of composition on compressive Young's moduli of 3D-printed allograft-PCL constructs. The 3D-printed constructs contained 0, 15 and 30 wt % allograft with dimensions: five layers of 10×10×0.5 mm; cylinder pore diameter: 0.5 mm. The moduli decreased with increasing the amount of allograft. n=6, *p<0.005.

FIGS. 5A-5B show that dental pulp stem cells had favorable attachment and viability in 3D-printed allograft-PCL constructs. In FIG. 5A, dental pulp stem cells seeded in the constructs showed nuclei localization along the filaments of allograft-PCL constructs by DAPI staining. In FIG. 5B the absorbance at 570 nm wavelength of dental pulp stem cells grown in the constructs containing 0, 15, and 30 wt % allograft, respectively, is measured for 24 h and treated with MTT. The absorbance increased with increasing the allograft content, indicating that the cells proliferated better with increasing the allograft content. n=4 *p<0.05.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure. For example, an osteoconductive allograft with a weight percent of about 5% to about 95% in a composite includes a weight percent of 4.5% to 97%.

As used herein, the terms “composite material”, “composite”, “hybrid bioregenerative composition”, and “hybrid material” are used interchangeably.

As used herein, the terms “bioresorbable construct” and “tissue or organ regenerative construct” are used interchangeably.

In one embodiment of the present invention, there is provided a composite material for regeneration of a tissue or an organ, comprising a mixture of a bioresorbable polymer and a bioresorbable graft material.

In this embodiment the tissue may be connective tissue, epithelial tissue, muscle, or nervous tissue; and wherein the organ is bone, cartilage, tendon, ligament, joint, tooth, nerve, blood vessel, artery, vein, capillary, lymphatic vessel, muscle, skin, heart, brain, skull, hypothalamus, cerebellum, kidney, liver, lung, ear, eye, cornea, lens, retina, vitreous, optic nerve, nose, olfactory epithelium, face, mouth, tongue, salivary gland, larynx, thymus gland, thyroid, trachea pancreas, spinal cord, stomach, small intestine, large intestine, cecum, colon, rectum, anus, genital, bladder, spleen, ureter, urethra, uterus, vagina, penis, scrotum, prostate, hair, teste, or nail.

In this embodiment, the bioresorbable polymer may be poly-caprolactone, polylactic acid, polylactic-co-glycolic acid, or polyethylene, pluronic acid (or polaxamer), polyesters, polyamides, polyurethane, polyorthoesters, polyanhydrides, polyethylene terephthalate, polycarbonates, polyfumarates, polycyanoacrylates, polyphosphazenes, polyphosphoesters, bioploymers, natural polymers, collagen, gelatin, elastin, elastin-like-peptides, fibrin, celluloses, chitosan, alginate (alginic acid), glycosaminoglycans, hyaluronic acid or silk, or a combination thereof.

In this embodiment, the bioresorbable graft material may be mineralized or demineralized or a combination thereof.

In this embodiment, the bioresorbable osteoconductive graft material may have a particle size of about 1 μm to about 250 μm.

In this embodiment, the bioresorbable osteoconductive allograft may have a weight percentage of about 5% to about 95% in the composite material.

In this embodiment, the bioresorbable osteoconductive graft material may be an allograft, an autograft or a xenograft. Particularly, the allograft is human demineralized tissue or organ matrix or animal demineralized tissue or organ matrix.

In another embodiment of the present invention there is provided a bioresorbable construct to repair a defect in a tissue or an organ, comprising a 3D structure printed from the composite material, as described supra.

Further to this embodiment the 3D structure further comprises a growth factor, a gene or an additive loaded therein with or without a biodegradable nanocarrier or cells, or a combination thereof. In another further embodiment, the 3D structure further comprises a sensor, an imaging probe, an imaging dye, or a semiconductor, or a combination thereof.

In all embodiments, the tissue and the organ are as described supra. Also in all embodiments the 3D structure may have dimensions corresponding to the defect in the tissue or the organ. In addition, the 3D structure may comprise a plurality of interconnected pores at about 5 μm to about 1000 μm in size. Furthermore, the 3D structure may have a Young's modulus that mimics that of bone, or other human or animal tissues or organs. Further still, the 3D structure may have a Young's modulus of about 1 KPa to about 300 MPa.

In all embodiments, representative growth factors include but are not limited to bone morphogenetic protein, bone morphogenetic protein-2, platelet derived growth factor, transforming growth factor, growth differentiation factor, insulin-like growth factor, multiplication-stimulating factor, vascular endothelial growth factor, fibroblast growth factor, nerve growth factor, neurotrophic factor, neurotrophin, hematopoietic growth factor, hepatocyte growth factor, erythropoietin, sarcoma growth factor, epidermal growth factor, granulocyte colony stimulating factor, granulocyte-macrophage colony-stimulating factor, thrombopoietin, or stem cell factor, or a combination thereof.

In all embodiments, the gene factors include but are not limited to RUNX2, OSX, SPARC, miR-142-5p, COL1A, BSP, OPN, miR-139-5p, ALP, OPG, miR-940, FHL2, NEUROG2, BRN, ASCL1, MYT1L, NEUROD1, miR-9, miR-124, LMX1A, FOXA2, LNX3, NURR1, PITX3, HB9, NGN1, NGN2, LSL1, ISL1, LHX3, Sema3a, Mapk8, Nrcam, Dlg4, Slit1, Creb1, Ntrk2, Cntn2, Pax6, Dcx, Nrcam, Ephb1, Sox7, Sox17, Sox18, Sox2, NANOG, NR5A2, DPPA3, E-cadherin, Myf5, MyoD, MRF4, and myogenin, c-Myc, p63, Lin28a, Oct3, Oct4, c-Myc or Klf-4, or a combination thereof.

In all embodiments, representative additives factors include but are not limited to calcium, tricalcium phosphate, magnesium, zinc, vitamin D, vitamin K, vitamin C, protein, osteopontin, osteocalcin, osteonectin, flavonoid, isoflavone, poly(aspartic acid), citrate, ceramic, metal, glass, titanium, hydroxyapetite acid, bone meal, antioxidant, probiotic or polyphenol, or a combination thereof.

In all embodiments, representative biodegradable nanocarrier factors include but are not limited to a nanoparticle, a nanomaterial, a nanocomposite, a micelle, a dendrimer, a liposome, a nanorod, a nanowire, a nanofiber, a nanotube, a quantum dot, a suspension, a dispersion, an emulsion, a membrane or a nanogel, or a combination thereof. Particularly, the biodegradable nanogel may comprise thermoresponsive poly(N-isopropylacrylamide) and biodegradable dextran-poly(lactate-2-hydroxyethyl-methacrylate) or dextran-poly(caprolactone-2-hydroxyethyl-methacrylate).

In all embodiments, representative cells factors include but are not limited to human cells or animal cells comprising bone (osteoblasts, osteoclasts, osteocytes), endothelial, nerve (neurons), neuroglial, muscle, red blood (erythrocytes), white blood cells [granulocytes (neutrophils, eosinophils, basophils), agranulocytes (monocytes, lymphocytes)], cartilage (chondrocytes), cardiac, smooth, epithelial, lining, skin (keratinocytes), fat (white adipocytes, brown adipocytes) or stem cells, or a combination thereof. Particularly, the stem cells may be human induced pluripotent stem cells, animal induced pluripotent stem cells, dental pulp, mesenchymal stem cells, progenitor stem cells, multipotent stem cells, oligopotent stem cells, totipotent stem cells, embryonic stem cells, fetal stem cells, adult stem cells, perinatal stem cells, neural stem cells, neural crest stem cells, hematopoietic stem cells, epithelial stem cells, endothelial stem cells, hepatic stem cells, adipose tissue-derived stem cells or muscle-derived stem cells, or a combination thereof.

In yet another embodiment of the present invention, there is provided a method for repairing a defect in a tissue or an organ subject in need thereof, comprising printing the bioresorbable construct of as described supra as the 3D structure comprising a plurality of interconnected pores thereon and with dimensions corresponding to the defect in the tissue or the organ defect; loading the 3D structure with stem cells, where the 3D structure optionally comprises a growth factor, a gene, an additive, tissue-specific cells, a sensor, an imaging probe, an imaging dye or a semiconductor, or a combination thereof; where the growth factor, the gene or the addition optionally is loaded into a biodegradable nanocarrier; and implanting the bioresorbable construct into the defect of the tissue or the organ, where the stem cells contained therein are induced to differentiate into tissue-specific cells, thereby regenerating the tissue or the organ in the subject.

In this embodiment, the tissue and the organ, the growth factor, the gene, the additive, the biodegradable nanocarrier, and the nanogel are as described supra. Also in this embodiment, the plurality of interconnected pores in the 3D structure may be about 5 μm to about 1000 μm in size.

In this embodiment, representative stem cells factors include but are not limited to human induced pluripotent stem cells, animal induced pluripotent stem cells, dental pulp, mesenchymal stem cells, progenitor stem cells, multipotent stem cells, oligopotent stem cells, totipotent stem cells, embryonic stem cells, fetal stem cells, adult stem cells, perinatal stem cells, neural stem cells, neural crest stem cells, hematopoietic stem cells, epithelial stem cells, endothelial stem cells, hepatic stem cells, adipose tissue-derived stem cells or muscle-derived stem cells, or a combination thereof.

In this embodiment, representative tissue-specific cells factors include but are not limited to human cells or animal cells comprising bone (osteoblasts, osteoclasts, osteocytes), endothelial, nerve (neurons), neuroglial, muscle, red blood (erythrocytes), white blood cells [granulocytes (neutrophils, basophils), eosinophils, agranulocytes (monocytes, lymphocytes)], cartilage (chondrocytes), cardiac, smooth, epithelial, lining, skin (keratinocytes), or fat (white adipocytes, brown adipocytes) or a combination thereof.

In yet another embodiment of the present invention, there is provided a method for preparing a composite of a bioresorbable polymer and a bioresorbable osteoconductive graft material suitable for 3D printing, comprising preparing a solid powder mixture of a resorbable polymer and an osteoconductive graft material; and milling the solid powder mixture to reduce a particle size of the osteoconductive graft material, thereby producing the composite. Further to this embodiment, the method comprises heating the composite to mold into pellets; and extruding the pellets into filaments suitable for 3D printing.

In both embodiments, representative resorbable polymer factors include but are not limited to poly-caprolactone, polylactic acid, polylactic-co-glycolic acid, or polyethylene, pluronic acid (or polaxamer), polyesters, polyamides, polyurethane, polyorthoesters, polyanhydrides, polyethylene terephthalate, polycarbonates, polyfumarates, polycyanoacrylates, polyphosphazenes, polyphosphoesters, bioploymers, natural polymers, collagen, gelatin, elastin, elastin-like-peptides, fibrin, celluloses, chitosan, alginate (alginic acid), glycosaminoglycans, hyaluronic acid or silk, or a combination thereof. Also, in both embodiments the graft material may be an allograft, an autograft or a xenograft.

In yet another embodiment of the present invention, there is provided a composite of a bioresorbable polymer and a bioresorbable graft material prepared by the method as described supra.

In this embodiment, the composite may comprise rpoly-caprolactone and human demineralized tissue or organ matrix. Also in this embodiment the human demineralized tissue or organ matrix may have a particle size of about 1 μm to about 250 μm and comprises about 5% weight percent to about 95% weight percent thereof.

In yet another embodiment of the present invention, there is provided a hybrid bioregenerative composition, comprising a composite of polycaprolactone and an allograft, an autograft, or a xenograft.

In this embodiment, the allograft may be demineralized. Particularly, the allograft may be human demineralized tissue or organ matrix or an animal demineralized tissue or organ matrix. Also in this embodiment, the allograft may be osteoconductive. In addition, the allograft may have a particle size of about 1 μm to about 250 μm. In addition, the allograft may have a weight percentage of about 5% to about 95% in the composite. Furthermore, the composite may be a powder or may comprise a plurality of filaments each formed for 3D printing.

In yet another embodiment of the present invention, there is provided a tissue or organ regenerative construct, comprising a composite of polycaprolactone and human demineralized tissue or organ matrix printed as a 3D structure with a plurality of interconnected pores and dimensions corresponding to a defect in the tissue or organ, each of said interconnected pores in the plurality comprising stem cells and, optionally, a growth factor, a gene, or an additive loaded with or without a biodegradable nanocarrier, tissue-specific cells, a sensor, an imaging probe, an imaging dye, or a semiconductor, or a combination thereof.

In this embodiment the human demineralized tissue or organ matrix may be about 5% to about 95% of the composite. Also in this embodiment, the interconnected pores may comprise the plurality are about 100 μm to about 500 μm in size. In addition the 3D structure may have a Young's modulus of about 1 KPa to about 300 MPa. In addition the tissue, the organ, the stem cells, the tissue-specific cells, the biodegradable nanocarrier, the nanogel, the growth factor, the gene, and the additive are as described supra.

In yet another embodiment of the present invention, there is provided a method for regenerating a tissue or an organ in a subject in need thereof, comprising implanting the tissue or organ regenerative construct as described supra into the defect in the tissue or organ, where the stem cells contained therein are induced to differentiate into tissue-specific cells, thereby regenerating the tissue or the organ in the subject.

The present invention combines biomaterials, nanotechnology, growth factors, stem cells and 3D-printing for regenerating complex tissues or organs. The present invention provides composite materials or composites and compositions of bioresorbable graft material and a bioresorbable polymer as hybrid materials that provide biorecognition and mechanical properties mimicking natural bone architecture and signal pathways to differentiate stem cells into tissue-specific cells to construct patient-specific and defect-site-specific tissue or organ grafts.

Generally, the graft material may be mineralized, demineralized or a combination thereof and may be a human or animal tissue or organ allograft, for example, human demineralized bone matrix or may be an autograft, or a xenograft. Preferably, the bioresorbable polymer may be polycaprolactone, polylactic acid, polylactic-co-glycolic acid, polyethylene, pluronic acid (or polaxamer), polyesters, polyamides, polyurethane, polyorthoesters, polyanhydrides, polyethylene terephthalate, polycarbonates, polyfumarates, polycyanoacrylates, polyphosphazenes, polyphosphoesters, bioploymers, natural polymers, collagen, gelatin, elastin, elastin-like-peptides, fibrin, celluloses, chitosan, alginate (alginic acid), glycosaminoglycans, hyaluronic acid or silk, or a combination thereof. More preferably, the bioresorbable polymer may be polycaprolactone.

Moreover, the present invention provides 3D bioresorbable constructs and tissue or organ regenerative constructs with a defined geometry and interconnected pores printed from the hybrid materials using extrusion based printers from Allevi and Prusa pneumatically and by FDM, respectively. The constructs may be printed with a 3-dimensional geometry that corresponds to a tissue or organ defect. The constructs have a mechanical strength better than any commercial synthetic organic substitutes and are close to that of native tissue or organ. The results showed that the compressive moduli of the constructs were between 1 KPa and 300 MPa depending on allograft amount and pore size. The compressive moduli decreased with increasing allograft amount from 0 to 30 wt % and pore size from 100 to 500 μm.

Furthermore, the 3D-constructs are useful for drug delivery for tissue or organ regeneration by using a biodegradable nanocarrier loaded with a growth factor, gene or additive to induce growth and differentiation of cells for tissue or organ repair. The nanocarrier factors include but are not limited to a nanoparticle, nanomaterial, nanocomposite, micelle, dendrimer, liposome, nanorod, nanowire, nanofiber, nanotube, quantum dot, suspension, dispersion, emulsion, membrane or nanogel, or a combination thereof. The nanogel may comprise thermoresponsive poly(N-isopropylacrylamide) and biodegradable dextran-poly(lactate-2-hydroxyethyl-methacrylate) or dextran-poly(caprolactone-2-hydroxyethyl-methacrylate). The constructs gradually and finally completely degrade on site after implantation and thus no polymers accumulate in the body and no surgical removal is required.

The following examples are given for the purpose of illustrating various embodiments of the invention and are not meant to limit the present invention in any fashion.

### Example 1

### Methods and Materials

### Preparation of Allograft and Polycaprolactone (PCL) Mixtures

Mixtures of bone allograft (LifeNet Health) and 50 kD MW polycaprolactone (Polysciences) are made in either 0, 10, 30, or 45 wt % allograft solid powder mixtures. Since allograft particles were too large to print, allograft was milled using a Planetary Ball Mill PM 100 (Retsch) at 270 rpm overnight to bring the allograft particle size down to tens of microns. The mixtures were either loaded onto a 5 ml syringe for 3D pneumatic extrusion printing using an Allevi 3D bioprinter, or fused deposition modeling (FDM) printing using a Prusa 3D printer. Before 3D printing using the Prusa 3D printer, varying mixtures of Allograft and PCL material were first formed into pellets by heating up the mixtures in 1 mL cylindrical molds at 90° C. and subsequently cooled into the shape of the mold. The released cylindrical composites were then fed into a Filastruder to convert the allograft-PCL material into 3D printable filaments for FDM printing.

### Pneumatic and FDM Extrusion 3D Printing Constructs Composed of Hybrid Allograft and PCL

A CAD model (Solidworks or FreeCAD) was used to design allograft-PCL constructs at dimensions of 1 mm×1 mm×0.5-2.5 mm, or 6 mm diameter×0.1-1 mm height. A nozzle diameter of 0.1-0.9 mm was used for the printing. For Allevi printing, the print parameters are: print temperature 90-100° C., bed temperature 35° C., print pressure 90-100 psi, and print speed 0.5-3 mm/s. For FDM printing, the print parameters are listed in Table 1.

TABLE 1
Print Parameters used for FDM Printing PCL on the Prusa Printer

| Printing Parameters | | |
|---|---|---|
| layer height | 0.25 | mm |
| extruder temp | 115 | C. |
| bed temp | 40 | C. |
| extrude width | 0.4 | mm |
| extrusion multiplier | 0.95 | |
| bridge flow ratio | 0.98 | |
| print speed (general) | 5 | mm/s |
| first layer speed | 2 | mm/s |
| bridge speed | 2.5 | mm/s |
| retract distance | 2.5 | mm |
| retract rate | 80 | mm/s |
| detract rate | 35 | mm/s |
| filament diameter (slicer) | 1.70 | mm |

| | |
|---|---|
| filament range | 1.6-1.85 |

### Mechanical Property of 3D-Printed Allograft-PCL Constructs

The compressive moduli of the printed allograft-PCL constructs were measured by using Instron 4505 using 5 kN load. Four to six constructs of each group are compressed to identify the materials' Young's modulus based on the slope of the initial stress-strain region.

### Cell Survival in 3D Printed Allograft-PCL Constructs

3D printed constructs containing 0, 10, 15, and 30 wt % allograft were assessed for dental pulp stem cell (DPSC) adhesion and viability. For adhesion studies, 10 wt % allograft constructs are seeded with 100,000 dental pulp stem cells and left on the constructs for a day. Cells on the constructs are stained with diamidino-2-phenylindole (DAPI) to view nuclei localization across the construct. For viability studies, 200,000 dental pulp stem cells are seeded into each construct containing 0, 15, or 30% allograft placed in 500 μL cell culture medium in 24 well plates. After 24 h, 100 μL of 3-(4,5-dimethyl-2-thiazolyl)-2,5-diphenyl-2-H-tetrazolium bromide (MTT) at 1.25 mg/mL was added to the well. After incubation at 37° C. for 4 h, the medium was removed from the well and DMSO was added in the well. The absorbance of the reduced form of the MTT was measured at 570 nm on a spark multimode microplate reader. Four replicate wells were used for each sample.

### Data Analysis and Statistics

Statistical comparisons are based on Student's t test or ANOVA with significance considered as p<0.05, and corrections for multiple comparisons when appropriate. When applicable, a two-way analysis of variance is performed and independent Student t tests are used to determine the significant differences between groups. Data is expressed as mean±S.D. or mean±S.E. of at least four separate experiments.

### Example 2

### Use of Nanogels to Control Insulin Release

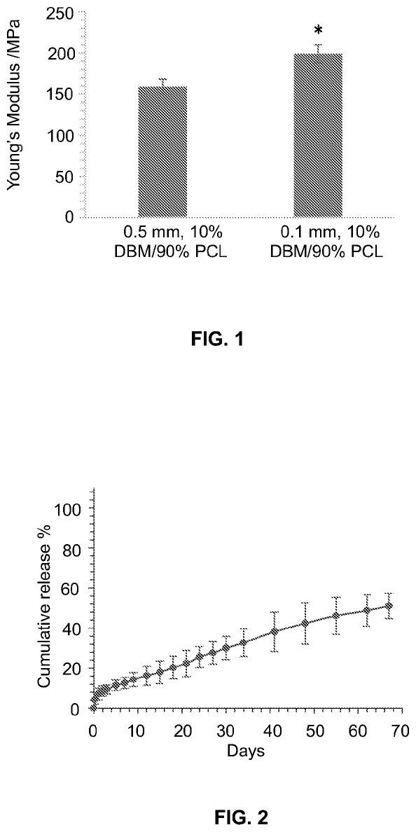

3D-printed constructs made of human DBM allograft and PCL composed of 10% allograft/90% PCL with 0.1 mm and 0.5 mm pores were printed using an Allevi 3 (3D Systems) bioprinter, we 3D-printed constructs. Dynamic Mechanical Analysis (DMA, Instron 5565) results (FIG. 1) showed that the 3D-printed constructs have high compression Young's modulus between 150 and 250 MPa comparable to some native bones. The modulus depended on the pore size; the higher was the pore size, the lower was the modulus.

Thermo-Responsive and Completely Biodegradable Nanogels are not Toxic and Prolong Model Insulin Release for More than 2 Months

Thermoresponsive and biodegradable nanogels were designed and synthesized by UV-emulsion polymerizing NIPAAm monomer and hydrolytically degradable DEXlactateHEMA macromer. These nanogels load insulin during the synthesis process in aqueous solution avoiding use of organic solvents which can denature antibodies. The insulin-loaded nanogels have a hydrodynamic diameter of 228.3±0.092 nm measured by dynamic light scattering (DLS) at 37° C. The nanogels could sustain the release of insulin for more than 2 months (FIG. 2). The nanogels were not cytotoxic to human dental pulp stem cells with above 90% cell viability at concentration up to 10 mg/mL after 24 h of incubation. The single-dose i.v. maximal tolerated dose (MTD) of the blank nanogels in NRG mice (n=3) was >800 mg/kg. These preliminary results demonstrate that the synthesized nanogels are not toxic in vitro and well tolerated by mice in vivo. The nanogels can sustain the release of proteins/peptides.

### Example 3

### Allograft-PCL Constructs

### Print Fidelity of Allograft-PCL Constructs

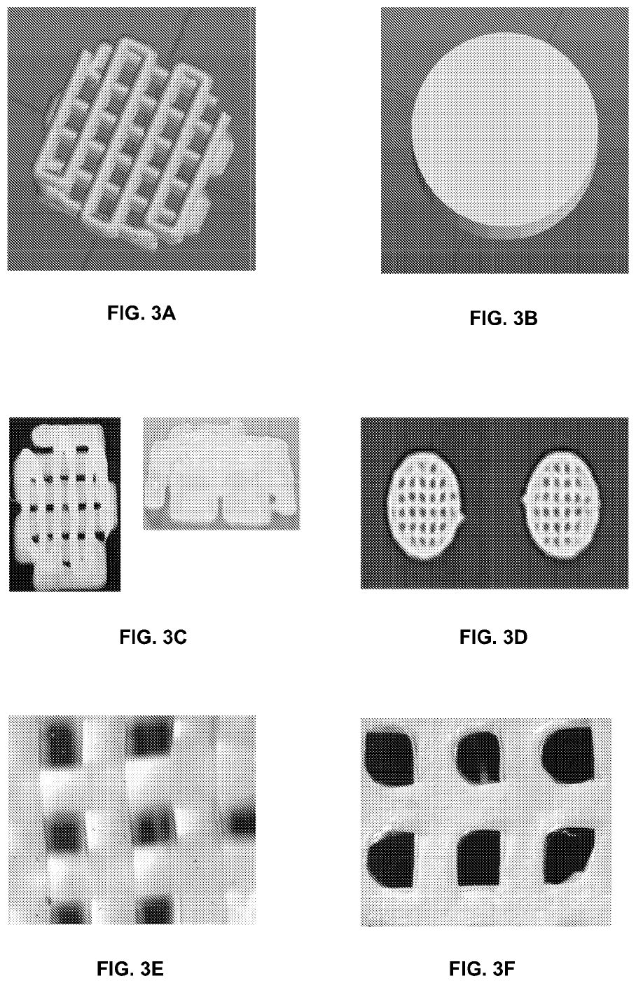

Constructs are measured by their diameters, heights, and/or pore sizes. For Allevi prints, the actual diameters, heights and pore sizes measured for interconnected pore constructs were between 5.82±0.09 mm to 5.86±0.07 mm, 0.78±0.11 to 0.86±0.11, and 400.89±46.83 to 463.24±34.38 μm, respectively which lie close to the theoretical dimensions. Cylindrical prints on Allevi were printed with as high as 45 wt % allograft with reported pore sizes of 614±72 μm. FDM prints using Prusa were only measured across their pores which are reported to have pore sizes of 580.12±33.81 μm. The resultant Allevi and FDM printed constructs are all diagrammed in FIGS. 3A-3F.

### Mechanical Property of 3D-Printed Allograft-PCL Constructs

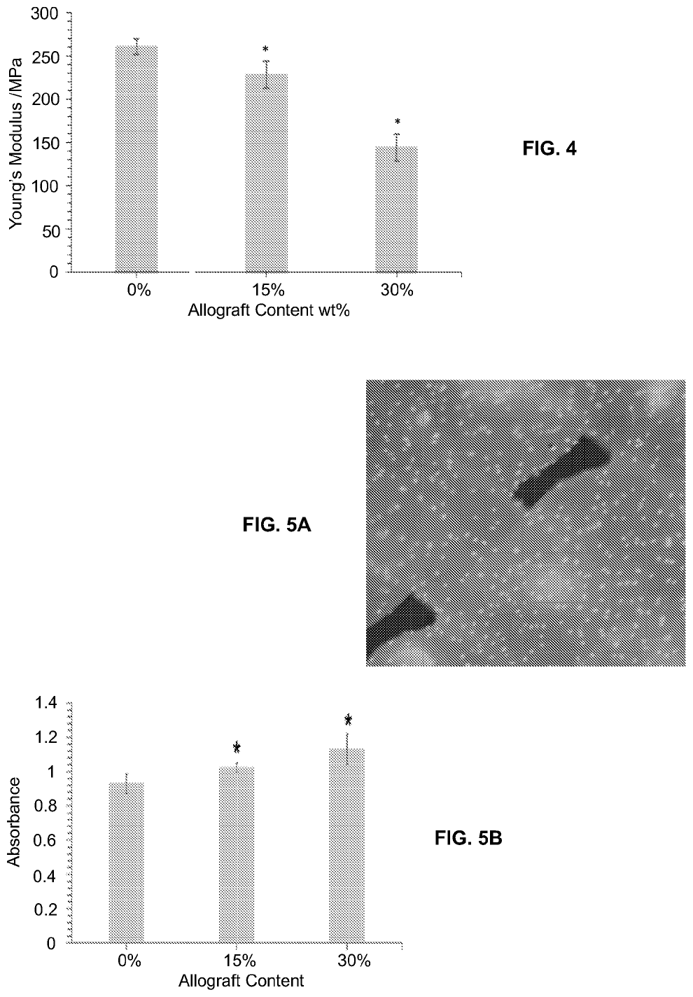

The Young's moduli of 3D-printed PCL-Allograft constructs with 0, 15 and 30 wt % allograft are diagrammed in FIG. 4. The compressive moduli of all the constructs were between 130 and 300 MPa, mimicking that of cortical bone (130-200 MPa). The Young's moduli decreased from 261.3±8.7 to 228.8±15.5 and 143.95±15.6 MPa when the composition of allograft was increased from 0 to 15 and 30 wt %, respectively.

### DPSC Survival in Allograft-PCL Constructs

The adhesion and the survival of dental pulp stem cells seeded in the 3D-printed allograft-PCL constructs were evaluated by DAPI-staining and MTT assay, respectively. In FIG. 5A, nuclei DAPI staining results showed that dental pulp stem cells readily attached onto the constructs. In FIG. 5B, MTT assay results showed that the optical absorbance of dental pulp stem cells was 0.929±0.058, 1.022±0.026, and 1.130±0.088 after the cells were grown in the constructs containing 0, 15, and 30 wt % allograft, respectively for 24 h and treated with MTT. These results suggested that dental pulp stem cells proliferated better with increasing allograft content due to the biorecognition nature of the allograft which contained demineralized bone components and natural proteins.

### In Vitro Characterization of Stemness Maintenance for Stem Cell Differentiation

A series of biodegradable 3D-constructs made of allograft and PCL with different weight ratios from 1:9 to 5:5, geometry 3-10×3-10×3-10 mm, and interconnected pore sizes from 100 to 1000 μm are printed using an Allevi-3 bioprinter. The 3-month hydrolytic degradation of the constructs is characterized by FTIR and weight loss. The porosity of the constructs with the hydrolytic degradation is measured by scanning electron microscopy (SEM). The rheological properties including storage and loss moduli, and mechanical compression modulus of the constructs is measured by rheometer and dynamic mechanical analyzer, respectively. The cytotoxicity of the constructs to DPSCs and iPSCs is evaluated by MTT and live-dead cell assays. The effects of the chemical, physical, rheological, mechanical and biodegradation properties of the constructs on the pluripotency of DPSCs/IPSCs and differentiation of DPSCs/iPSCs into bone-forming cells is assessed by measuring stemness gene expression, and osteoblast markers and mineralization, respectively.

In Vitro Differentiation of Stem Cells into Osteoblasts

To add additional controlled chemical cues for the stem cells to grow and differentiate, nanogels are developed to control and sustain the release of osteogenic growth factors BMP-2 and PDGF-BB within the 3D-printed allograft-PCL constructs. The nanogels are made of thermoresponsive PNIPAAm and biodegradable DEXPlactateHEMA with different hydrophobicity, hydrophilicity, crosslinker amount and length, surface charge and particle size. BMP-2 and/or PDGF-BB is loaded into the nanogels during the nanogel synthesis in aqueous solution as described (21). The chemical structure, size, zeta-potential, and degradation of the nanogels is characterized by FTIR, DLS/TEM/AFM, zeta-potential, and FTIR, respectively.

The release of BMP-2 and/or PDGF-BB from the nanogels is carried out in PBS (pH 7.4) at 37° C. for three months and quantified by UPLC and mass spectroscopy. Mathematical models quantitatively describe the in vitro BMP-2/PDGF-BB release kinetics. The cytotoxicity of the nanogels to dental pulp stem cells and iPSCs is evaluated by MTT assay. The in vitro effects of the 3D-printed constructs with and without the BMP-2 and PDGF-loaded nanogels on the stemness and osteoblastic differentiation of DPSCs/iPSCs is examined.

In Vivo Bone Formation after Implantation of Nanogel-Encapsulated Hydrogels

A 5-6 mm-calviarie-defect rat model (22,23) is created and DPSC/iPSC- and BMP-2/PDGF-BB-loaded nanogels-encapsulated 3D-printed constructs are implanted at the defect site. Bone regeneration is measured in the healing site 8 weeks after implantation by micro-CT 3D image analysis and bone histology. Real-time RT-PCR and western blot analysis are performed for bone-related gene expressions and compared with the naïve control groups and the control groups with the 3D-construct, stem cells, BMP2/PDGF-BB, and nanogels only or 2-3 combinations thereof.

The following references are cited herein.
1. Wu et al. Lancet Healthy Longevity, 2 (9): E580-E592, 2021.
2. Salari et al. Journal of Orthopaedic Surgery and Research, 16 (1), 2021.
3. Roddy et al. European Journal of Orthopaedic Surgery and Traumatology, 28 (3): 351-362, 2018.
4. Bezstarosti et al. Archives of Orthopaedic and Trauma Surgery, 141 (7): 1215-1230, 2021.
5. Bone Grafts And Substitutes Market Size, Share & Trends Analysis Report By Material Type (Allograft, Synthetic), By Application (Spinal Fusion, Foot & Ankle, Joint Reconstruction), By Region, And Segment Forecasts, 2022-2030. Research Reports in Medical Devices, 2022;
6. Perez et al. Frontiers in Bioengineering and Biotechnology, 6, 2018.
7. Hosseini et al. Journal of Dental Research, 100 (10): 1011-1019, 2021.
8. Qu et al. Rsc Advances, 9 (45): 26252-26262, 2019.
9. Kashirin et al. Biomaterials Science, 7 (10): 3961-3983, 2019.
10. de Grado et al. Journal of Tissue Engineering, 9, 2018.
11. Wang WH and Yeung KWK. Bioactive Materials, 2 (4): 224-247, 2017.
12. Zhang PB and Zhu QS. Regenerative Biomaterials, 2 (2): 153-158, 2015.
13. Oryan et al. Journal of Orthopaedic Surgery and Research, 9, 2014.
14. Zhang et al. Frontiers in Bioengineering and Biotechnology, 8, 2020.
15. Wilson WC and Boland T. Anatomical Record Part a—Discoveries in Molecular Cellular and Evolutionary Biology, 2003, 272A (2): 491-496, 2003.
16. Roque et al. Journal of Manufacturing Processes, 2021, 64:655-663, 2021.
17. Nulty et al. Acta Biomaterialia, 126:154-169, 2021.
18. Genova et al. International Journal of Molecular Sciences, 21 (19), 2020.
19. Ashammakhi et al. Advanced Healthcare Materials, 8 (7), 2019.
20. Murphy SV and Atala A. Nature Biotechnology, 32 (8): 773-785, 2014.
21. Huang et al. Macromolecules, 41 (22): 8339-8345, 2008.
22 Dang et al. Applied Materials Today, 20, 2020.
23 Pryor et al. Journal of Periodontology, 2005, 76 (8): 1287-1292, 2005.

1: A composite material for regeneration of a tissue or an organ, comprising:
a mixture of a bioresorbable polymer and a bioresorbable graft material.

2: The composite material of claim 1, wherein the tissue is connective tissue, epithelial tissue, muscle, or nervous tissue; and wherein the organ is bone, cartilage, tendon, ligament, joint, tooth, nerve, blood vessel, artery, vein, capillary, lymphatic vessel, muscle, skin, heart, brain, skull, hypothalamus, cerebellum, kidney, liver, lung, ear, eye, cornea, lens, retina, vitreous, optic nerve, nose, olfactory epithelium, face, mouth, tongue, salivary gland, larynx, thymus gland, thyroid, trachea pancreas, spinal cord, stomach, small intestine, large intestine, cecum, colon, rectum, anus, genital, bladder, spleen, ureter, urethra, uterus, vagina, penis, scrotum, prostate, hair, teste, or nail.

3: The composite material of claim 1, wherein the bioresorbable polymer is polycaprolactone, polylactic acid, polylactic-co-glycolic acid, polyethylene, pluronic acid (or polaxamer), polyesters, polyamides, polyurethane, polyorthoesters, polyanhydrides, polyethylene terephthalate, polycarbonates, polyfumarates, polycyanoacrylates, polyphosphazenes, polyphosphoesters, bioploymers, natural polymers, collagen, gelatin, elastin, elastin-like-peptides, fibrin, celluloses, chitosan, alginate (alginic acid), glycosaminoglycans, hyaluronic acid or silk, or a combination thereof.

4: The composite material of claim 1, wherein the bioresorbable graft material is mineralized or demineralized or a combination thereof.

5: The composite material of claim 1, wherein the bioresorbable graft material has a particle size of about 1 μm to about 250 μm.

6: The composition of claim 1, wherein the bioresorbable allograft has a weight percentage of about 5% to about 95% in the composite material.

7: The composite material of claim 1, wherein the bioresorbable graft material is an allograft, an autograft or xenograft.

8: The composite material of claim 7, wherein the allograft is human demineralized tissue or organ matrix or animal demineralized tissue or organ matrix.

9: A bioresorbable construct to repair a defect in a tissue or an organ, comprising:
a 3D structure printed from the composite material of claim 1.

10: The bioresorbable construct of claim 9, wherein the tissue is connective tissue, epithelial tissue, muscle, or nervous tissue; and wherein the organ is bone, cartilage, tendon, ligament, joint, tooth, nerve, blood vessel, artery, vein, capillary, lymphatic vessel, muscle, skin, heart, brain, skull, hypothalamus, cerebellum, kidney, liver, lung, ear, eye, cornea, lens, retina, vitreous, optic nerve, nose, olfactory epithelium, face, mouth, tongue, salivary gland, larynx, thymus gland, thyroid, trachea, pancreas, spinal cord, stomach, small intestine, large intestine, cecum, colon, rectum, anus, genital, bladder, spleen, ureter, urethra, uterus, vagina, penis, scrotum, prostate, hair, teste, or nail.

11: The bioresorbable construct of claim 9, wherein the 3D structure has dimensions corresponding to the defect in the tissue or the organ.

12: The bioresorbable construct of claim 9, wherein the 3D structure comprises a plurality of interconnected pores at about 5 μm to about 1000 μm in size.

13: The bioresorbable construct of claim 9, wherein the 3D structure has a Young's modulus that mimics that of bone, or other human or animal tissues or organs.

14: The bioresorbable construct of claim 9, wherein the 3D structure has a Young's modulus of about 1 KPa to about 300 MPa.

15: The bioresorbable construct of claim 9, wherein the 3D structure further comprises a growth factor, a gene or an additive loaded therein with or without a biodegradable nanocarrier or cells, or a combination thereof.

16-23. (canceled)

24: A method for repairing a defect in a tissue or an organ in a subject in need thereof, comprising:
printing the bioresorbable construct of claim 9 as the 3D structure comprising a plurality of interconnected pores thereon and with dimensions corresponding to the defect in the tissue or the organ defect;
loading the 3D structure with stem cells, said 3D structure optionally comprising a growth factor, a gene, an additive, tissue-specific cells, a sensor, an imaging probe, an imaging dye or a semiconductor, or a combination thereof; said growth factor, said gene or said addition optionally loaded into a biodegradable nanocarrier; and
implanting the bioresorbable construct into the defect of the tissue or the organ, said stem cells contained therein induced to differentiate into tissue-specific cells, thereby regenerating the tissue or the organ in the subject.

25: The method of claim 24, wherein the tissue is connective tissue, epithelial tissue, muscle, or nervous tissue; and wherein the organ is bone, cartilage, tendon, ligament, joint, tooth, nerve, blood vessel, artery, vein, capillary, lymphatic vessel, muscle, skin, heart, brain, skull, hypothalamus, cerebellum, kidney, liver, lung, ear, eye, cornea, lens, retina, vitreous, optic nerve, nose, olfactory epithelium, face, mouth, tongue, salivary gland, larynx, thymus gland, thyroid, trachea, pancreas, spinal cord, stomach, small intestine, large intestine, cecum, colon, rectum, anus, genital, bladder, spleen, ureter, urethra, uterus, vagina, penis, scrotum, prostate, hair, teste, or nail.

26: The method of claim 24, wherein the plurality of interconnected pores in the 3D structure are about 5 μm to about 1000 μm in size.

27: The method of claim 24, wherein the stem cells are human induced pluripotent stem cells, animal induced pluripotent stem cells, dental pulp, mesenchymal stem cells, progenitor stem cells, multipotent stem cells, oligopotent stem cells, totipotent stem cells, embryonic stem cells, fetal stem cells, adult stem cells, perinatal stem cells, neural stem cells, neural crest stem cells, hematopoietic stem cells, epithelial stem cells, endothelial stem cells, hepatic stem cells, adipose tissue-derived stem cells or muscle-derived stem cells, or a combination thereof.

28: The method of claim 24, wherein the tissue-specific cells are human cells or animal cells comprising bone (osteoblasts, osteoclasts, osteocytes), endothelial, nerve (neurons), neuroglial, muscle, red blood (erythrocytes), white blood cells [granulocytes (neutrophils, eosinophils, basophils), agranulocytes (monocytes, lymphocytes)], cartilage (chondrocytes), cardiac, smooth, epithelial, lining, skin (keratinocytes), or fat (white adipocytes, brown adipocytes) or a combination thereof.

29: The method of claim 24, wherein the growth factor is bone morphogenetic protein, bone morphogenetic protein-2, platelet derived growth factor, transforming growth factor, growth differentiation factor, insulin-like growth factor, multiplication-stimulating factor, vascular endothelial growth factor, fibroblast growth factor, nerve growth factor, neurotrophic factor, neurotrophin, hematopoietic growth factor, hepatocyte growth factor, erythropoietin, sarcoma growth factor, epidermal growth factor, granulocyte colony stimulating factor, granulocyte-macrophage colony-stimulating factor, thrombopoietin, or stem cell factor, or a combination thereof.

30: The method of claim 24, wherein the gene is RUNX2, OSX, SPARC, miR-142-5p, COL1A, BSP, OPN, miR-139-5p, ALP, OPG, miR-940, FHL2, NEUROG2, BRN, ASCL1, MYT1L, NEUROD1, miR-9, miR-124, LMX1A, FOXA2, LNX3, NURR1, PITX3, HB9, NGN1, NGN2, LSL1, ISL1, LHX3, Sema3a, Mapk8, Nrcam, Dlg4, Slit1, Creb1, Ntrk2, Cntn2, Pax6, Dcx, Nrcam, Ephb1, Sox7, Sox17, Sox18, Sox2, NANOG, NR5A2, DPPA3, E-cadherin, Myf5, MyoD, MRF4, and myogenin, c-Myc, p63, Lin28a, Oct3, Oct4, c-Myc or Klf-4, or a combination thereof.

31: The method of claim 24, wherein the additive is calcium, tricalcium phosphate, magnesium, zinc, vitamin D, vitamin K, vitamin C, protein, osteopontin, osteocalcin, osteonectin, flavonoid, isoflavone, poly(aspartic acid), citrate, ceramic, metal, glass, titanium, hydroxyapetite acid, bone meal, antioxidant, probiotic, or polyphenol, or a combination thereof.

32: The method of claim 24, wherein the biodegradable nanocarrier is a nanoparticle, a nanomaterial, a nanocomposite, a micelle, a dendrimer, a liposome, a nanorod, a nanowire, a nanofiber, a nanotube, a quantum dot, a suspension, a dispersion, an emulsion, a membrane or a nanogel, or a combination thereof.

33: The method of claim 32, wherein the nanogel comprises thermoresponsive poly(N-isopropylacrylamide) and biodegradable dextran-poly(lactate-2-hydroxyethyl-methacrylate) or dextran-poly(caprolactone-2-hydroxyethyl-methacrylate).

34-60. (canceled)
